(19) United States
(12) Patent Application Publication
(10) Pub. No.: US 2012/0316644 A1
(43) Pub. Date: Dec. 13, 2012

(54) INTRAOCULAR LENS IMPLANT FIXATION

(76) Inventor: Kenneth J. Wald, Scarsdale, NY (US)

(21) Appl. No.: 13/158,720

(22) Filed: Jun. 13, 2011

(51) Int. Cl. A61F 2/16

(52) U.S. Cl. 623/6.43

(57) ABSTRACT

An intraocular lens fixation device including a deformable, medically inert clip that affixes a haptic of an intraocular lens to a patient's iris. A method of affixing an intraocular lens, having haptics, within a patient's eye includes piercing the iris in at least two locations that closely flank a portion of one of the haptics, inserting an arm of a biologically inert deformable clip through each of the piercings in the iris until a distal end of each arm is located on opposing sides of the haptic, and applying a force to each of the arms to deploy the clip by causing the distal ends of the arms to approach each other and substantially surround and constrain both a section of the iris that is between the arms and a portion of the haptic.

### BACKGROUND

1. Field

This disclosure relates generally to intraocular lenses, and, more particularly, to intraocular lens fixation.

2. Background

Intraocular lens implants (“IOLs”) are commonly used to replace the natural lens of the human eye when warranted by medical conditions, such as cataracts. These processes typically involve the removal of the natural lens from the capsular bag through the cornea of the eye, and the subsequent insertion of the IOL into the capsular bag. The IOLs are usually stabilized within the capsular bag by haptic arms that extend outward from the IOL. In some cases, the IOLs may later become dislocated due to, for example, problems with the capsular bag or the zonules that hold it in place, or due to a malpositioned haptic. As a result, a second surgery is necessary to reposition the dislocated lens and may require suturing of the IOL in place through the iris.

### BRIEF SUMMARY

In one aspect of this disclosure, an intraocular lens fixation device is disclosed for affixing a portion of an intraocular lens haptic to an iris of an eye. The device comprises a clip made of a biologically inert, deformable material. The clip includes at least two spaced apart arms extending from a back portion of the clip, the spaced apart arms at their distal ends prior to deployment being slightly wider than the portion of the intraocular lens haptic. Each arm has a length that exceeds a combined thickness of the portion of the intraocular lens haptic and the iris at a location where the arm passes through the iris. The clip being positioned with the back portion on a side of the iris opposite the haptic portion and a substantial part of the length of the arms being on a haptic-side of the iris with the arms each being located on opposing sides of the portion of the intraocular lens haptic and deformed such that (i) the distal ends of the arms are spaced narrower than the portion of the intraocular lens haptic, and (ii) the back portion and arms collectively surround and constrain, within an interior surface of the clip, both a section of the iris and the portion of the haptic, thereby affixing the iris and intraocular lens haptic to each other.

In another aspect of this disclosure, an intraocular lens fixation system is disclosed. The system comprises a clip including at least two arms, the clip being made of a biologically inert, deformable material. The arms of the clip are spaced apart at their ends so as to form a gap that is slightly larger than a portion of an intraocular lens haptic that will be affixed to an iris of an eye using the clip. A microforcep tip having a pair of opposed jaws each including distal ends, the jaws being configured to hold the clip therebetween. Either (a) the ends of the arms, (b) distal ends of the jaws, or (c) both, are sharpened to facilitate piercing of the iris to either side of the portion of the haptic. In addition, the jaws and clip are aligned relative to each other to allow for piercing of the iris and insertion of the arms of the clip through the iris such that when the arms of the clip have passed through the pierced iris, the arms will flank the portion. The jaws of the microforcep tip are further configured to be able to apply a compressive force to the arms sufficient to cause the clip to deform such that the ends of the arms on a side of the haptic opposite the iris move towards each other and thereby cause the clip to affix the haptic to the iris at the portion.

In yet another aspect of this disclosure, a method of affixing an intraocular lens within an eye of a patient is disclosed. The intraocular lens includes at least two haptics located on a side of the patient's iris opposite the cornea. The method comprises piercing the iris in at least two locations that closely flank a portion of one of the haptics. An arm of a biologically inert deformable clip is inserted through each of the piercings in the iris until a distal end of each arm is located on opposing sides of the portion of one of the haptics. A force is applied to each of the arms so as to deploy the clip by causing the distal ends of the arms to approach each other and substantially surround and constrain, within an interior surface of the clip, both a section of the iris that is between the arms and the portion of one of the haptics.

The foregoing and following discussion outlines rather generally the features and technical advantages of one or more embodiments of this disclosure in order that the following detailed description may be better understood. Additional features and advantages of this disclosure will be described herein and may be the subject of claims of this application.

### BRIEF DESCRIPTION OF THE DRAWINGS

This disclosure is further described in the detailed description that follows, with reference to the drawings, in which:

FIG. 1 illustrates, in simplified form, a cross section of a typical human eye;

FIGS. 2 and 3 respectively illustrate, in simplified form, an eye in which the lens has been removed and replaced with an IOL;

FIGS. 4A and 4B illustrate, in simplified form, an illustrative clip suitable for use in securing a haptic to the iris as described herein;

FIG. 5 illustrates, in simplified form, another illustrative clip similar to the clip of FIG. 4;

FIG. 6A illustrates, in simplified form, another illustrative clip that includes a recess or other discontinuity on the interior surface;

FIG. 6B illustrates, in simplified form, another illustrative clip that includes a recess or other discontinuity on the upper surface of the back portion;

FIG. 7A illustrates, in simplified form, another illustrative clip with multiple recesses or discontinuities;

FIG. 7B illustrates, in simplified form, the illustrative clip of FIG. 7A deformed into the deployed configuration;

FIG. 8 illustrates, in simplified form, one example implementation of a clip deploying component;

FIG. 9 illustrates, in simplified form, an alternative example implementation of a clip deploying component;

FIG. 10 illustrates, in simplified form, an illustrative deployment device;

FIGS. 11A through 11E illustrate, in simplified form, an example of the process used to affix an IOL haptic to the iris using the devices and components described herein;

FIGS. 12A and 12B respectively illustrate, in simplified form, the eye and IOL of FIGS. 2 and 3 following deployment of two clips as described herein to affix the IOL within the eye by affixing the haptics to the iris.

### DETAILED DESCRIPTION

In simplified overview, to avoid the time consuming and difficult suture-based approach to fixation of IOL haptics to the iris, the instant disclosure describes an approach whereby a clip is deployed through the iris that affixes the haptic to the iris. The clip is made deformable so that, once arms of the clip are inserted through the iris, the clip can be deformed in place and thereby affix a haptic of the IOL to the iris in a way analogous to the way a staple can hold multiple pieces of paper together.

Implementations of the device and method described herein can provide advantages over many conventional methods for correcting dislocated IOLs. For example, some implementations of the disclosed device and method enable repair of a dislocated IOL merely by repositioning of the IOL (i.e., without requiring removal of the old dislocated IOL). Similarly, some implementations will be superior to suturing the haptic arms to the iris because suturing is much more difficult to perform, less durable over time, and carries much greater risk for younger patients. In contrast, implementations of the methods described herein are relatively easy to perform and require far less skill than suturing approaches, and implementations of the device can be much more durable than suturing approaches.

The above will become evident from the following description.

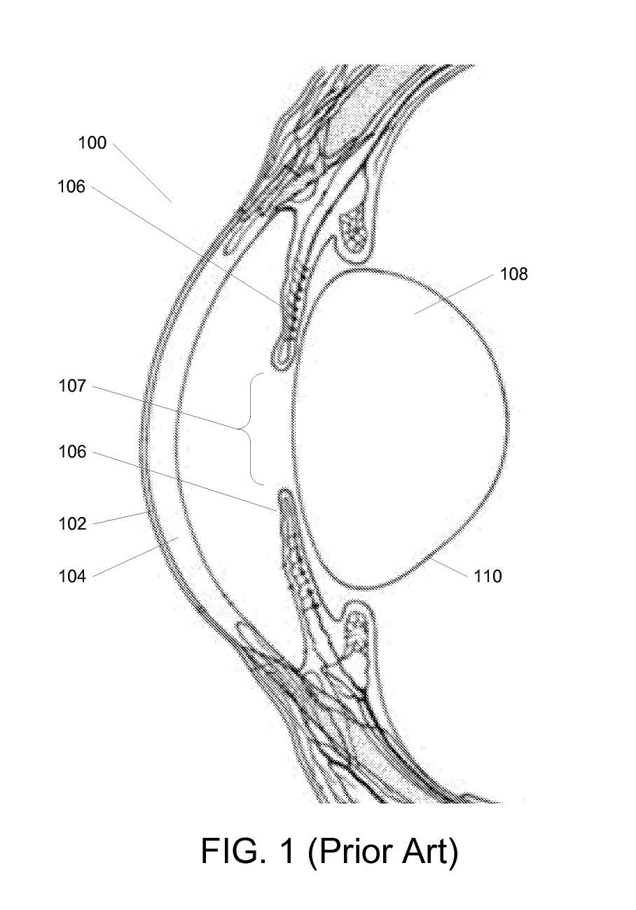

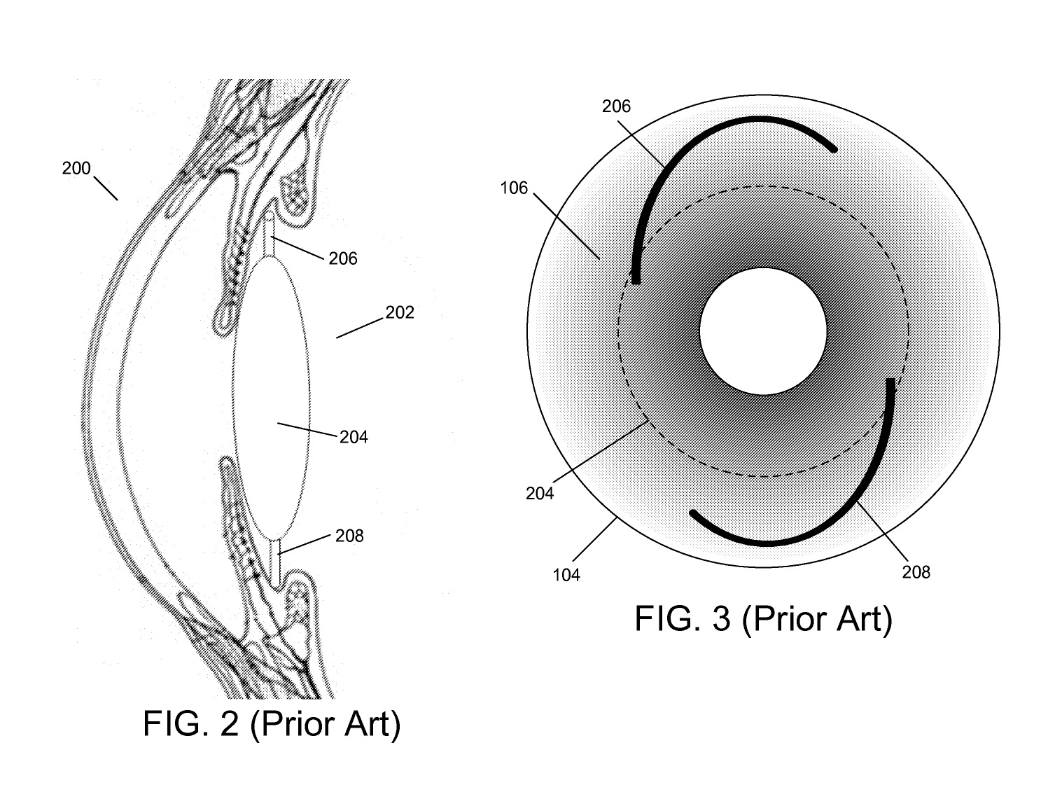

FIG. 1 illustrates, in simplified form, a cross section of a typical human eye 100, which includes (among other components) the conjunctiva 102, the cornea 104, the iris 106, which defines the pupil opening 107, and a lens 108 having an outer skin called the capsule 110. When cataract surgery is performed, the lens 108 is removed and replaced with an artificial lens generally referred to as an intraocular lens (IOL). Depending upon the particular issues and surgery, replacement of the lens 108 with an IOL may or may not leave the capsule 110 intact. IOLs come in different configurations, and the type of IOL pertinent to the instant disclosure is one such as shown in FIGS. 2 and 3, which respectively illustrate, in simplified form, an eye 200 in which the lens has been removed and replaced with an IOL 202 of the type made up of a IOL lens 204 and two or more curved and outwardly extending arms 206, 208, generally referred to as “haptics,” that suspend the IOL lens 204 behind the iris 106, as well as stabilize and maintain the IOL 202 in place within the eye 200. FIG. 2 is a cutaway side view similar to FIG. 1, and FIG. 3 is a simplified forward view along the direction “A” of FIG. 2.

In instances where an IOL 202 becomes dislodged, repositioning alone may not be sufficient to avoid another dislocation in the future. Moreover, in some cases, the internal eye structures necessary to support the haptics 206, 208 may be damaged or otherwise incapable of doing so.

In such cases, an approach and device described herein permits the IOL 202 to be repositioned and used without the need for removal of the IOL or the difficulty associated with the current iris suture technique.

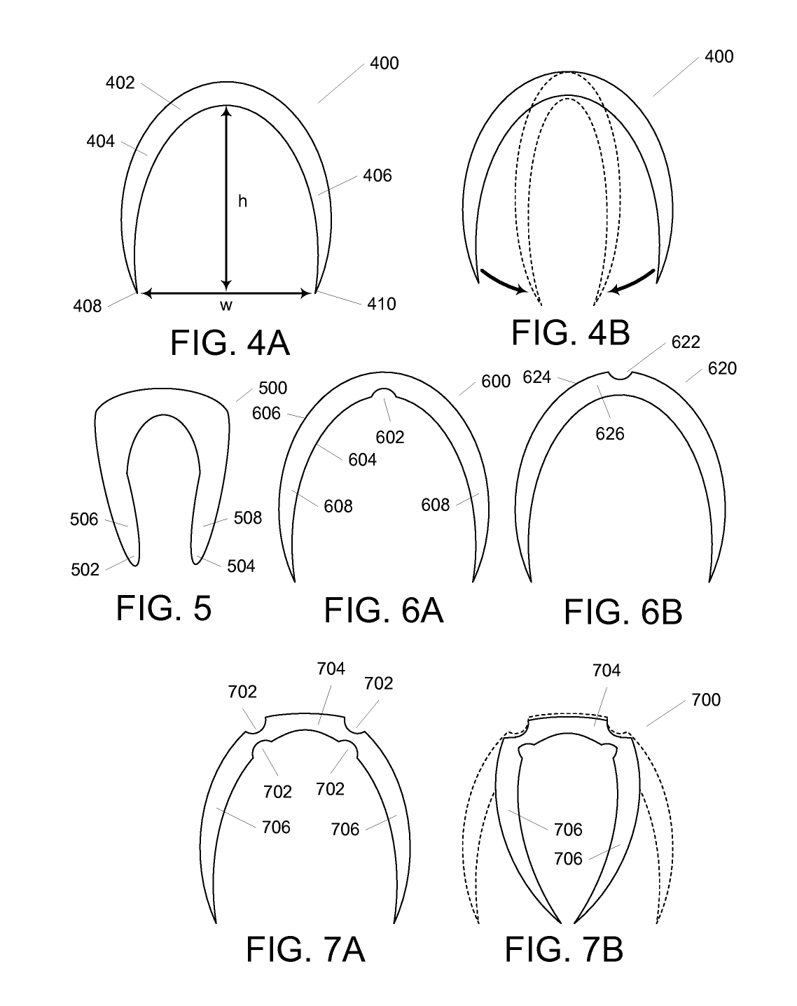

FIGS. 4A and 4B illustrate, in simplified form, a clip 400 that can be used for this purpose. The clip 400 is preferably configured in a “U,” “C” or staple-like shape comprising a back portion 402 and two arms 404, 406, which, depending upon the particular implementation, may be blunt or sharpened at their distal ends 408, 410. The clip 400 is made of a deformable and medically inert material that is safe for use inside the eye and will not degrade or oxidize within the eye and that, once positioned, can be compressed to a new position, such as shown in FIG. 4B and described in greater detail below, and will retain its shape thereafter (i.e., once deployed). Suitable deformable materials may include (but are not limited to) polymethyl-methacrylate (PMMA), a silicone or polypropylene coated deformable material, other acrylics, as well as other non-metal medically inert materials with this same characteristic (i.e., deformable and retains shape thereafter). Advantageously, use of a clear polymer such as PMMA renders the clip less visible on the iris. Moreover, use of a non-metal deformable material provides further advantages in that it will not be affected by procedures involving, for example, magnetic resonance imaging (MRI) or use of energy or radiation that would be absorbed by and adversely heat the metal and thereby damage the iris, the IOL haptic or both. In addition, the clip is sized so that the overall spacing w between the distal ends 408, 410 is sufficiently wide so as to be wider than the IOL haptic 206, 208 at the location where it will be deployed, typically between about 1 mm and about 2 mm, but not so wide as would allow the IOL to be displaced to the extent that the effectiveness of the IOL in use is compromised. Similarly, the interior depth h is sized to be sufficient to allow for both the relevant portion of the IOL haptic and a piece of the iris to be non-destructively contained within the interior surface of the clip when the distal ends 408, 410 are brought together as described below such that, after deployment, they are spaced apart at a distance that is sufficiently less than the width of the IOL haptic 206, 208 at the location where it will be deployed to thereby ensure good and long-lasting fixation, typically less than about 2.5 mm.

FIG. 5 illustrates, in simplified form an alternative clip 500 that is similar to the clip 400 of FIG. 4, but differs in that the distal ends 502, 504 of the arms 506, 508 of the clip 500 are blunt instead of sharpened.

As noted above, deployment of a clip requires that the clip be deformed to a new position, which, as noted above, will serve to affix a haptic of an IOL to the iris. As a result, depending upon the particular implementation, as well as the material used for the clip, it may be desirable to specifically control the location(s) where the deformation takes place. This can be accomplished by, for example, weakening or otherwise pre-deforming a part of the clip in such a manner as will result in the clip deforming uniformly and/or in those areas first. FIGS. 6A, 6B, 7A and 7B illustrate, in simplified form, such a feature.

For example, FIG. 6A illustrates, in simplified form, a clip 600 that includes a recess or other discontinuity 602 on the interior surface 604 of the clip 600 rendering it thinner in the middle such that the application of a force to the exterior surface 606 of the arms 608 will cause the clip 600 to more easily bend near the recess 602. FIG. 6B illustrates, in simplified form, a clip 620, similar to the clip 600 of FIG. 6A, except that the recess or discontinuity 622 is located on the upper surface 624 of the back portion 626 of clip 620.

FIG. 7A illustrates, in simplified form, another illustrative clip 700, that is similar to the aforementioned clips 600, 620, except that the clip 700 includes multiple recesses or discontinuities 702 located on the clip 700 so that the back portion 704 of the clip 700 does not deform when a force is applied as described herein and the arms 706 deflect inward when the clip 700 is deformed from the configuration of FIG. 7A to the deployed configuration shown in FIG. 7B.

Depending upon the particular implementation used for the clip, the recess(es) or discontinuity/(ies) that act as a deformation control crease can be created by cutting, molding, forging, deforming, etc. Alternatively, any other known way of controlling or confining bending of a solid within a prescribed area, which will still allow the clip to perform its intended purpose, can be used. Similarly, different shaped clips can be used, the clips of FIGS. 4A, 4B, 5, 6A, 6B, 7A and 7B being merely illustrative of the intended concept.

Having described various aspects and illustrative clips suitable for use with the approach described herein, a device for the deployment of a clip will now be described.

As noted above, deployment of a clip requires essentially two capabilities: (1) an ability to pierce the iris, and (2) an ability to deform the clip when appropriately positioned.

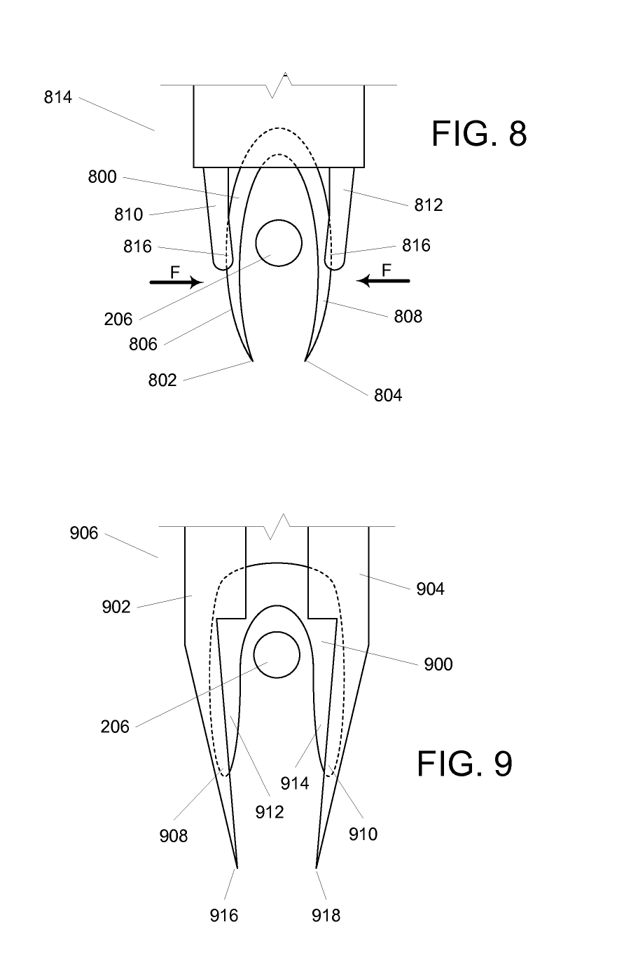

FIG. 8 illustrates, in simplified form, one example implementation suitable for meeting both these objectives and, thus, deploying a clip as described herein. As shown in FIG. 8, a clip 800, similar to the clips discussed above and which has sharpened distal ends 802, 804 on the arms 806, 808 of the clip 800, is contained within a pair of jaws 810, 812 at the distal end of a microforcep tip 814, which will be described in greater detail below and only part of which is shown. With the arrangement of FIG. 8, the first capability is provided by the sharpened distal ends 802, 804 and the second capability is provided by the jaws 810, 812. In addition, as shown, the jaws 810, 812 each include a recess, groove or indentation 816 to receive and constrain one arm 806, 808 of the clip 800 prior to, and while, the clip 800 is being deployed. The jaws 810, 812 are configured to be movable towards each other and apply a force to the arms 806, 808 of the clip 800 in the directions “F” and thereby deform the clip by moving the distal ends 802, 804 on the arms 806, 808 of the clip 800 towards each other.

FIG. 9 illustrates, in simplified form, another illustrative implementation suitable for meeting both the above objectives and, thus, deploying a clip as described herein. As shown in FIG. 9, and similar in configuration and operation to FIG. 8, a clip 900 is contained within a pair of jaws 902, 904 of a microforcep tip 906 (only part of which is shown). However, in this illustrative embodiment, the distal ends 908, 910 of the arms 912, 914 of the clip 900 are blunt (similar to that shown in clip 500 of FIG. 5), but the distal ends 916, 918 of the jaws 902, 904 of the microforcep tip 906 are sharpened. Thus, in this embodiment, the jaws 902, 904 have both required capabilities of piercing the iris and deforming the clip.

In general, the maximum width of the microforcep tip with the clip contained therein, measured along the portion that will be introduced into the eye, should be less than the width of a sutureless corneal incision, which is generally about 2.75 mm wide, to ensure that it can be used in connection with such incisions. Of course, larger incisions will allow for larger microforcep tip widths, even if such a corneal incision would require suture closure.

It is worthwhile noting that, although less desirable, the distal ends of both the clip and microforcep tip can be blunt. However, with such blunt embodiments, some other means of piercing the iris as described herein (for example a needle) will be necessary and the complexity and risk associated with the procedure will be greater. Nevertheless, such variants are contemplated as being within the scope of the subject matter described herein.

In general, a microforcep tip is essentially a modified version of a conventional microforcep or microscissor, specifically one where, in the case of a microforcep, the jaws are modified or grooved to hold the clip and allow for sufficient but not excessive force to be transferred to the arms of a clip as described herein to meet the above requirements for deploying the clip. In the case of a microscissor, the blades may be made thicker and the cutting surfaces dulled to preclude slicing of the iris tissue and an appropriate recess or other form of constraining the clip prior to and during deployment would be included therein.

Microforcep or microscissors suitable for modification to accomplish deployment of a clip as described herein are commercially available from numerous sources including, for example: Accutome Inc., 3222 Phoenixville Pike, Malverne, Pa. 19355; Alcon Laboratories Inc., 6201 South Freeway, Fort Worth Tex. 76134; ASICO LLC, 26 Plaza Drive, Westmont, Ill. 60559; Bausch & Lomb Surgical, 30 Enterprise, Suite 450, Aliso Viejo, Calif. 92656; Dutch Opthalmic USA, 10 Continental Drive, Exeter, N.H. 03833; Geuder AG, Hertzstrasse 4, 69216 Heidelberg, Germany; and Pelion Surgical, 1525 Sunshine Court, Aiken, S.C. 29803.

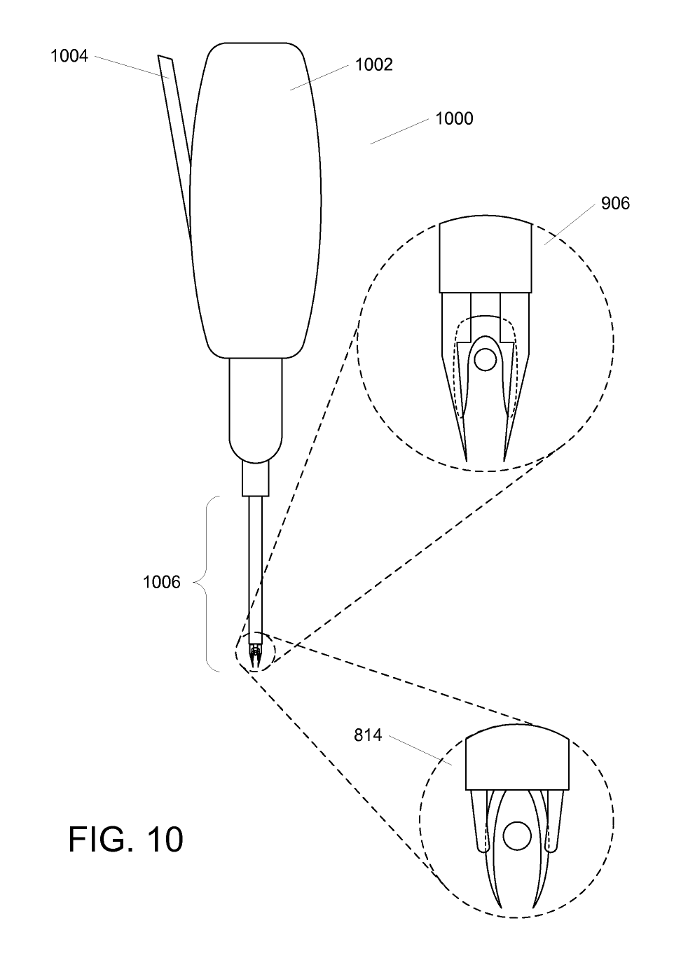

In addition, in some implementations, the microforcep tip is a permanent part of a deployment device, whereas with other implementations, the microforcep tip is attachable/detachable. FIG. 10 illustrates, in simplified form, an illustrative deployment device 1000. The deployment device 1000 includes, for example, a conventional microforcep or microscissors body such as that used with single use/disposable microforceps or microscissors that generally includes a handle 1002 and a lever or other mechanism 1004 (along with appropriate internal components) of appropriate conventional design through which the jaws (such as those shown in FIG. 8 or FIG. 9) can be actuated and thereby transmit a force applied by a user (with appropriate force scaling if necessary) to the jaws of the microforcep tip 1006.

Advantageously, where the microforcep tip 1006 is removable, the microforcep tip 1006 can be separately provided in sterile packaging with the clip pre-loaded in a single-use configuration. Alternatively, the components can be provided such that the clip must be loaded into the microforcep tip 1006 and properly oriented within the jaws prior to use. In such a case, given the sizing involved, the groove or recess can be specifically shaped such that when the clip is inserted it snaps into place with the proper alignment.

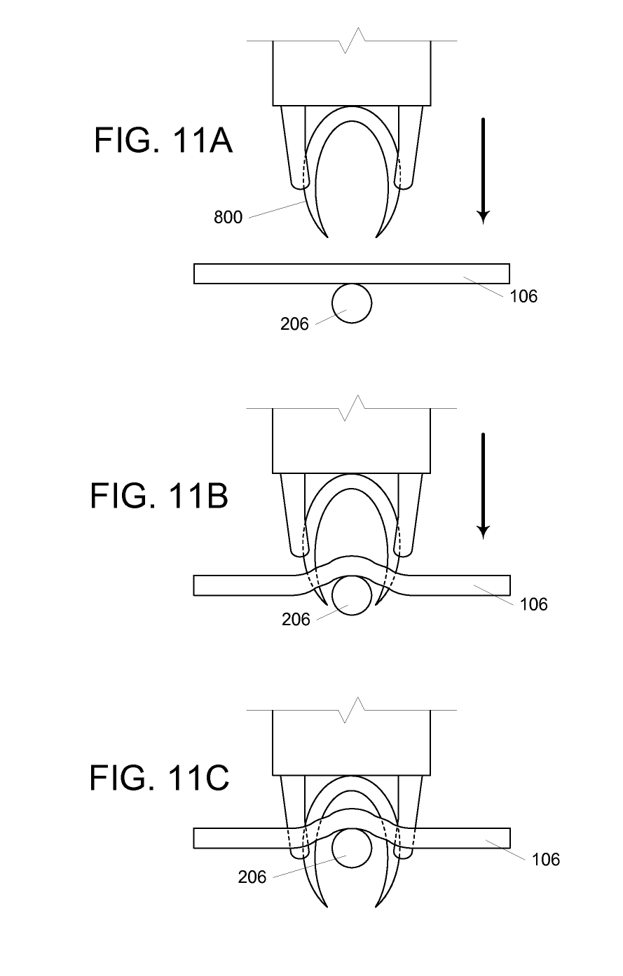

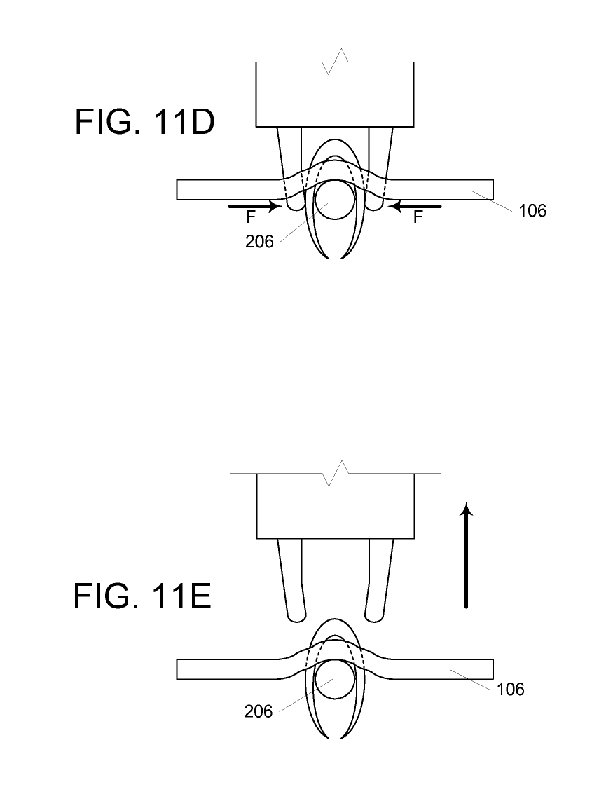

Having generally described the various components, a preferred iris fixation process will now be described with reference to FIGS. 11A through 11E, which includes a simplified representation of part of the iris tissue and corresponding portion of a haptic of an IOL.

To perform the affixation, the surgeon first locates a haptic arm 206 of the IOL and makes sure that it is positioned appropriately underneath the iris 106 in, for example, the conventional manner as would be used for a suturing affixation technique. Then, the clip 800 is positioned above the iris 106 and the portion of the haptic arm 206 to be used to secure the IOL such as shown in FIG. 11A. Next, the portion of iris 106 above the haptic arm 206 is pierced to either side of the haptic 206 such that each arm of the clip flanks one side of the haptic portion as shown in FIG. 11B. The clip is inserted until the distal ends of the clip are at a sufficient depth that assures that when the distal ends of the clip are brought together, they will be clear of the haptic 206 and the portion of the haptic will be fully contained within a volume defined by the interior surface of the clip such as shown in FIG. 11C. At this point, a force is applied by the surgeon to the appropriate actuator on the handle of the deployment device (not shown) so as to cause the jaws of the device to apply a force to the clip, for example, in the directions “F” and thereby permanently deform the clip into its final position such as shown in FIG. 11D. At this point, the clip is fully deployed and the force can be released and the microforcep tip can be withdrawn leaving the clip in place securely fastening the haptic 206 to the iris 106 as shown in FIG. 11E.

The process can then be repeated with a new clip for each additional IOL haptic that has not yet been secured to the iris 106 as described above.

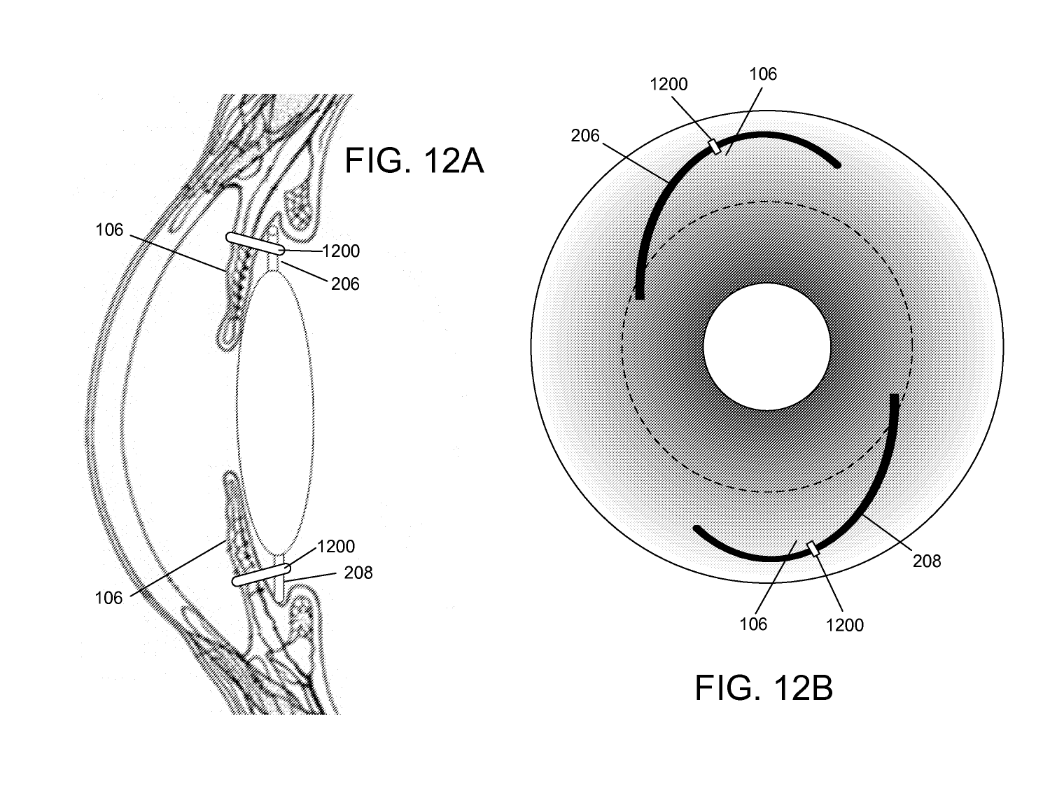

FIGS. 12A and 12B respectively illustrate, in simplified form, the eye and IOL of FIGS. 2 and 3 following deployment of two clips 1200 as described herein to affix the IOL within the eye by affixing the IOL haptics 206, 208 to the iris 106. As can be seen in the cutaway side view of FIG. 12A and the forward view of FIG. 12B, the clips 1200 pass through the iris 106 with the arms of each clip 1200 flanking a portion of one haptic 206, 208 thereby securing the haptics 206, 208 to the iris 106 and thereby securing and stabilizing the IOL in the proper position within the eye, even if the capsular bag cannot provide support or the zonules are damaged.

It should be understood that this description (including the figures) is only representative of some illustrative embodiments. For the convenience of the reader, the above description has focused on a representative sample of all possible embodiments, a sample that teaches the principles of the invention. The description has not attempted to exhaustively enumerate all possible variations. That alternate embodiments may not have been presented for a specific portion of the invention, or that further undescribed alternate embodiments may be available for a portion, is not to be considered a disclaimer of those alternate embodiments. One of ordinary skill will appreciate that many of those undescribed embodiments incorporate the same principles of the invention as claimed and others are equivalent.

What is claimed is:

1. An intraocular lens fixation system, comprising:
a clip including at least two arms, the clip being made of a biologically inert, deformable material, the arms of the clip being spaced apart at their ends so as to form a gap that is slightly larger than a portion of an intraocular lens haptic that will be affixed to an iris of an eye using the clip;
a microforcep tip having a pair of opposed jaws each including distal ends, the jaws being configured to hold the clip therebetween;
wherein either (a) the ends of the arms, (b) distal ends of the jaws, or (c) both, are sharpened to facilitate piercing of the iris to either side of the portion of the haptic, and wherein the jaws and clip are aligned relative to each other to allow for piercing of the iris and insertion of the arms of the clip through the iris such that when the arms of the clip have passed through the pierced iris, the arms will flank the portion, and wherein the jaws of the microforcep tip are further configured to be able to apply a compressive force to the arms sufficient to cause the clip to deform such that the ends of the arms on a side of the haptic opposite the iris move towards each other and thereby cause the clip to affix the haptic to the iris at the portion.

2. The intraocular lens fixation system of claim 1, wherein the material comprises one of a plastic, an acrylic, polymethyl-methylacrylate, a silicone covered material, or polypropylene covered material.

3. The intraocular lens fixation system of claim 1, wherein the material of the clip and a material for the haptic are the same.

4. The intraocular lens fixation system of claim 1, wherein the clip further comprises a deformation control crease.

5. The intraocular lens fixation system of claim 1, wherein the clip is substantially C-shaped.

6. The intraocular lens fixation system of claim 1, wherein the clip is substantially U-shaped.

7. The intraocular lens fixation system of claim 1, wherein the clip is substantially staple-shaped.

8. The intraocular lens fixation system of claim 1, further comprising a microforcep handle coupled to an end of the microforcep tip opposite the jaws.

9. The intraocular lens fixation system of claim 8, wherein the microforcep tip is a single-use component that is attachable to and detachable from the microforcep handle.

10. The intraocular lens fixation system of claim 1, wherein each of the jaws comprises a recess that substantially corresponds to a surface of an arm of the clip.

11. A method of affixing an intraocular lens within an eye of a patient, the intraocular lens having at least two haptics located on a side of the patient's iris opposite the cornea, the method comprising:
(a) piercing the iris in at least two locations that closely flank a portion of one of the haptics;
(b) inserting an arm of a biologically inert deformable clip through each of the piercings in the iris until a distal end of each arm is located on opposing sides of the portion of one of the haptics; and
(c) applying a force to each of the arms so as to deploy the clip by causing the distal ends of the arms to approach each other and substantially surround and constrain, within an interior surface of the clip, both a section of the iris that is between the arms and the portion of one of the haptics.

12. The method of claim 11, wherein “(a)” and “(b)” occur substantially concurrently.

13. The method of claim 11, wherein “(c)” further comprises deforming the clip at about a predetermined location.

14. The method of claim 11, further comprising loading the clip into a compressive tool.

15. The method of claim 11, further comprising attaching a microforcep tip having the clip loaded therein into a microforcep control handle.

16. The method of claim 15, wherein, following “(c)”, the method comprises:
withdrawing the microforcep tip from the eye while leaving the clip deployed; and
detaching the microforcep tip from the microforcep control handle.

17. The method of claim 15, further comprising loading the clip into the microforcep tip such that the arms of the clip are aligned with jaws of the microforcep tip.

18. The method of claim 11, wherein the piercing is performed by sharpened distal ends of the arms.

19. The method of claim 11, wherein the piercing is performed by sharpened jaws of the microforcep tip.

20. The method of claim 11, further comprising repeating “(a)” through “(c)” for another one of the haptics.

21. An intraocular lens fixation device for affixing a portion of an intraocular lens haptic to an iris of an eye, comprising:
a clip made of a biologically inert, deformable material, the clip including at least two spaced apart arms extending from a back portion of the clip, the spaced apart arms at their distal ends prior to deployment being slightly wider than the portion of the intraocular lens haptic, each arm having a length that exceeds a combined thickness of the portion of the intraocular lens haptic and the iris at a location where the arm passes through the iris, the clip being positioned with the back portion on a side of the iris opposite the haptic portion and a substantial part of the length of the arms being on a haptic-side of the iris with the arms each being located on opposing sides of the portion of the intraocular lens haptic and deformed such that (i) the distal ends of the arms are spaced narrower than the portion of the intraocular lens haptic, and (ii) the back portion and arms collectively surround and constrain, within an interior surface of the clip, both a section of the iris and the portion of the haptic, thereby affixing the iris and intraocular lens haptic to each other.

22. The intraocular lens fixation device according to claim 21, wherein a transition between the arms and the back portion of the clip is smooth and continuous.

23. The intraocular lens fixation device according to claim 21, wherein the clip includes a discrete and distinct indicator of a transition between the arms and the back portion.

24. The intraocular lens fixation device according to claim 21, wherein the distal ends of the clip are sharpened.

25. The intraocular lens fixation device according to claim 21, wherein the distal ends of the clip are blunt.

26. The intraocular lens fixation device of claim 21, wherein the biologically inert, deformable material comprises one of a plastic, an acrylic, polymethyl-methylacrylate, a silicone covered material, or polypropylene covered material.

27. The intraocular lens fixation device of claim 21, wherein the material of the clip and a material for the haptic are the same.

28. The intraocular lens fixation device of claim 21, wherein the clip further comprises a deformation control crease.

29. The intraocular lens fixation device of claim 21, wherein, prior to deployment, the clip is substantially C-shaped.

30. The intraocular lens fixation device of claim 21, wherein, prior to deployment, the clip is substantially U-shaped.

31. The intraocular lens fixation device of claim 21, wherein, prior to deployment, the clip is substantially staple-shaped.
